(19) United States
(12) Patent Application Publication
(10) Pub. No.: US 2021/0023111 A1
(43) Pub. Date: Jan. 28, 2021

(54) Novel Form of Invermectin and a Process for Making it

(71) Applicant: Hovione Scientia Limited, Cork (IE)

(72) Inventor: Sérgio SILVA, Lisboa (PT)

(21) Appl. No.: 16/980,489

(22) PCT Filed: Mar. 19, 2019
(86) PCT No.: PCT/GB2019/050760

§ 371 Date: Sep. 14, 2020

(30) Foreign Application Priority Data
Mar. 19, 2018 (PT) 110634

(51) Int. Cl. A61K 31/7048; A61K 9/16

(52) U.S. Cl. CPC A61K 31/7048; A61K 9/1688; A61K 9/16

(57) ABSTRACT

Amorphous ivermectin is provided, suitably in isolated solid form, and is suitably free of any additives or a support matrix, such as a solid dispersion. Also provided is a pharmaceutical formulation, for animal including human or veterinary use, comprising the amorphous ivermectin of the invention, and a pharmaceutically-acceptable carrier therefor. Also provided is a medical device incorporating amorphous ivermectin according to the invention, or a medical device incorporating a pharmaceutical formulation according to the invention described herein. A method of preparing amorphous ivermectin comprises the steps of preparing a solution of ivermectin in at least one solvent; removing the solvent by feeding the solution to a spray dryer and collecting particles of ivermectin. Amorphous ivermectin as disclosed herein may be used as a medicament, in particular to treat conditions such as a medical condition caused by internal nematode infections including but not limited to onchocerciasis (river blindness), filariasis (elephantiasis), strongyloidiasis or demodicosis.

### CROSS REFERENCE TO RELATED APPLICATIONS

This application is a filing under 35 U.S.C. 371 of International Application No. PCT/GB2019/050760 filed Mar. 19, 2019, entitled “A Novel Form of Invermectin and a Process for Making it,” which claims priority to Portuguese Patent Application No. 110634 filed Mar. 19, 2018, both of which are incorporated by reference herein in their entirety.

### FIELD OF INVENTION

The present invention relates in general to a novel form of ivermectin, and to a process for making it. Pharmaceutical formulations comprising the novel form, and medical devices incorporating it, are also disclosed. The novel form may be used as a medicament to treat a range of medical conditions.

The present invention relates to a new stable amorphous form of ivermectin and a method of producing stable amorphous ivermectin particles by dissolving ivermectin in a suitable solvent or mixture of solvents, optionally purifying the solution and isolating particles of essentially amorphous ivermectin by spray drying. Particularly, the present invention relates to stable amorphous ivermectin in a particulate form, with particle size in the micrometer and sub-micrometer range, and their uses. Also particularly, the present invention relates to a method of producing stable amorphous ivermectin in a particulate form, with particle size in the micrometer and sub-micrometer range, and their uses. The method can be applied in the pharmaceutical field particularly in the preparation of novel formulations ivermectin. The method enables the introduction of an absolute filtration step immediately before the final product isolation, removing foreign particles and controlling microbial and endotoxin contamination of the final API. Moreover, the amorphous particulates produced in accordance with the method of present invention present advantageous characteristics regarding purity, particle size, density, color and solubility.

Ivermectin is a semi-synthetic substance derived from avermectin, which is naturally produced by Streptomyces avermitilis and is a potent antiparasitic, and particularly anthelmintic, agent that is useful against a broad spectrum of endoparasites and ectoparasites in mammals as well as having agricultural uses against various parasites found in and on crops and in soil. Ivermectin is disclosed in U.S. Pat. No. 4,199,569, issued 22 Apr. 1980 to Chabala and Fisher (also EP0045655 (A2)).

It is very effective in the control of endo- and ecto-parasites such as gastrointestinal nematodes, lice, and mites in livestock (Camargo et al., 2010). Since its commercial introduction for the treatment of parasitic diseases in animals in the 1980s, over 5 billion doses of ivermectin have been sold worldwide making it the most commonly used antiparasitic drug in animals (Omura, 2008). Ivermectin is still used to treat billions of livestock and pets around the world and increases the production of meat and leather products. Ivermectin is also used for the treatment of parasitic diseases in humans. Since it was first approved to treat onchocerciasis (river blindness) in humans in 1988, ivermectin has been used worldwide to treat a variety of internal nematode infections including onchocerciasis, filariasis (elephantiasis), and strongyloidiasis as well as ectoparasitic infections by lice and mites. However, the oral bioavailability of ivermectin is very low—namely in ruminants. This is because of its poor water solubility, binding to organic materials in the gastro-intestinal tract, and transport by the P-glycoprotein present in the intestinal epithelium. As a result, ivermectin is generally administered subcutaneously to maximize its bioavailability. A few drug delivery technologies and systems have been used to develop the injectable ivermectin formulation (Rothen-Weinhold and Dahn, 2000; Dong et al., 2014), including sustained release injectable ivermectin formulation through solid dispersions.

We have appreciated that an amorphous form of ivermectin in a particulate form with controlled particle size could provide certain advantages. To the best of our knowledge, ivermectin has never been reported to be isolated in a stable amorphous form. However, despite this, we have now managed to provide such a form.

Ivermectin is a mixture, in the ratio of approximately 80:20 of 22,23-dihydroavermectin B1a and B1b.

U.S. Pat. No. 6,265,571 describes a process for purifying the bacterial fermentation product ivermectin.

Ivermectin solid dispersions are disclosed in:

Ivermectin-loaded microparticles for parenteral sustained release: in vitro characterization and effect of some formulation variables (J Microencapsul. 2010; 27(7):609-17)

Sustained release ivermectin-loaded solid lipid dispersion for subcutaneous delivery: in vitro and in vivo evaluation (Drug Deliv, 2017; 24(1): 622-631).

There is no prior art describing a stable form of amorphous ivermectin or a process to obtain amorphous form ivermectin without the use of additives or of a support matrix (solid dispersion).

In a broad aspect, the present invention provides amorphous ivermectin. The amorphous ivermectin is suitably in isolated form, meaning that the amorphous ivermectin suitably consists of, or consists essentially of, ivermectin alone, without needing any further compound or additive to provide stability. The amorphous invermectin is suitably free of any additives or a support matrix, such as a solid dispersion.

The amorphous form of ivermectin disclosed herein is of particular utility owing to its stability and its modified solubility profile.

The present invention also provides a pharmaceutical formulation, for animal including human or veterinary use, comprising amorphous ivermectin as described herein, and a pharmaceutically-acceptable carrier therefor.

In a further aspect, the invention provides a medical device incorporating amorphous ivermectin according to the invention described herein, or a medical device incorporating a pharmaceutical formulation according to the invention described herein.

In a further aspect, the invention provides a method of preparing amorphous ivermectin, which method comprises the steps of preparing a solution of ivermectin in at least one solvent; removing the solvent by feeding the solution to a spray dryer and collecting particles of ivermectin.

The present invention also provides amorphous ivermectin according to the invention described herein one, or a pharmaceutical formulation according to the invention described herein, or a medical device according to the invention described herein, for use as a medicament.

### BRIEF DESCRIPTION OF THE FIGURES

FIG. 1 shows the molecular structure of ivermectin.

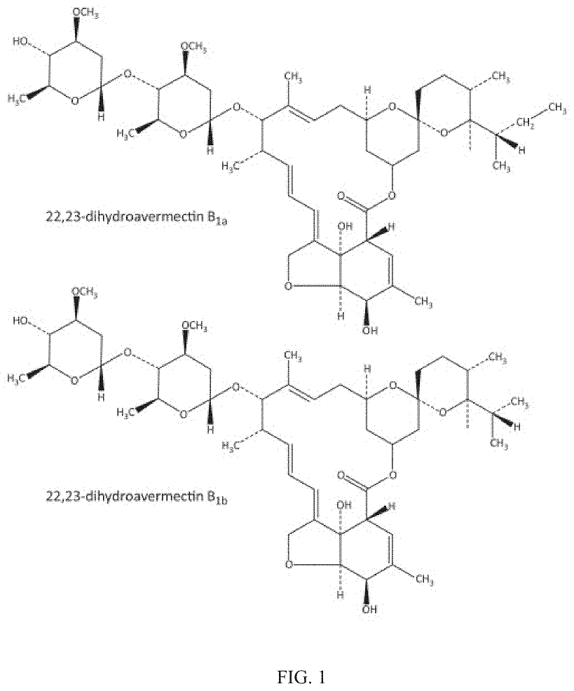

FIG. 2 shows XRP diffractogram of ivermectin obtained by spray drying.

FIG. 3 shows the DSC curves of amorphous ivermectin.

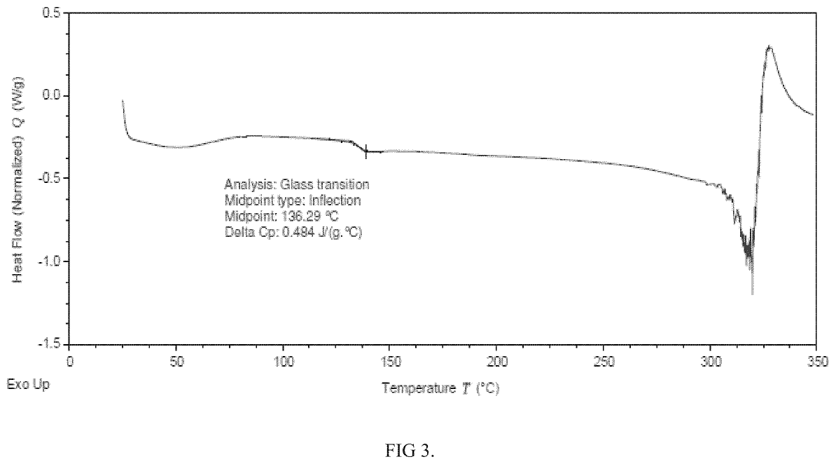

FIG. 4 schematic of a spray drying set up which may be used.

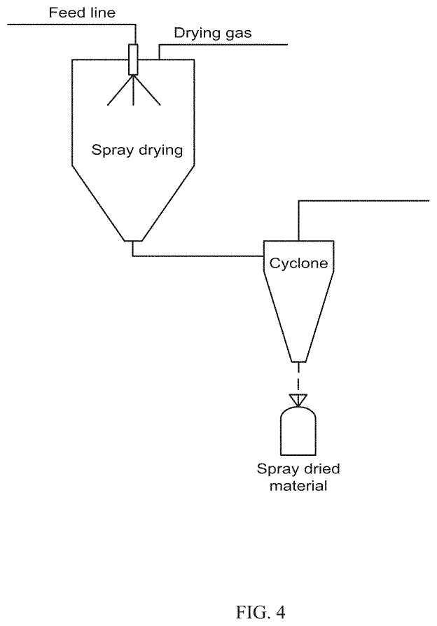

FIG. 5 representative SEM image of spray dried particles of ivermectin.

FIG. 6 representative histogram of the particle size distribution of amorphous ivermectin obtained as per example 1.

FIG. 7 shows the XRP diffractogram of ivermectin obtained by spray drying before and after storage for 3 months in the freezer plus 3.5 months at room temperature.

### DETAILED DESCRIPTION

Although the compound ivermectin has been known for many years, the present inventors have recognized that, in fact, no stable solid amorphous form of drug-alone ivermectin has ever been described. The present invention provides a novel amorphous solid form of ivermectin, which we have found can, quite surprisingly, be obtained by a simple and industrial method comprising spray drying of a solution of ivermectin. In one aspect of this invention the amorphous solid form of ivermectin is stable at room temperature. In another aspect of this invention the amorphous solid form of ivermectin is isolated in micrometer and nanometer size particulates.

In one aspect of this invention, amorphous ivermectin is suitable to be used in animal, including human, and veterinary pharmaceutical applications. In another aspect of this invention, amorphous ivermectin is suitable to be used in topical, oral, injectable, ophthalmic and inhaled formulations. In another aspect of this invention, amorphous ivermectin is suitable to be used in or administered by medical devices.

In a preferred aspect, the amorphous ivermectin is provided solid form. Preferably, the solid form is solid at room temperature (25° C.). Preferably, the amorphous ivermectin is in particulate form, especially in the form of particles having a mean diameter of 4 μm or less (as measured by laser diffraction), or wherein the particles have a mean diameter of from 0.1 μm to 4 μm. Particles obtained or obtainable by spray drying are preferred. Spherical, or substantially spherical, particles are highly preferred.

In a highly preferred aspect of the invention, the amorphous ivermectin is not present as, or is free from, a solid dispersion; for example, not present as, or free from, a solid dispersion with a polymer or a lipid. In a preferred aspect, the amorphous ivermectin is “drug-alone”—that is, free of any other additives apart from the ivermectin itself and any residual solvent(s) used during manufacture. Residual solvent at levels in accordance with ICH guidelines is acceptable, as is well understood in the pharmaceutical field. It will be understood that amorphous ivermectin which is “drug-alone” or consists of amorphous ivermectin may additionally comprise such residual solvent.

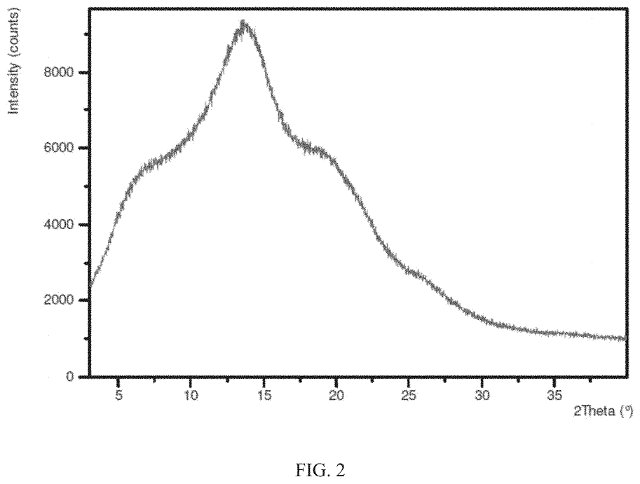

The invention provides amorphous ivermectin characterized by a broad XRPD pattern typical of amorphous material, wherein the XRPD pattern has no sharp diffraction peaks. Suitably, amorphous ivermectin is provided characterized by having an XRPD pattern as shown in, or substantially as shown in, FIG. 2.

The invention also provides amorphous ivermectin characterized by a glass transition temperature as measured by differential scanning calorimetry (DSC) of above 100° C. Suitably, the amorphous ivermectin is characterized by a glass transition temperature as measured by differential scanning calorimetry (DSC) of about 136° C.

The amorphous ivermectin according to invention is preferably provided in particulate form. This can be achieved, for example, by spray drying. Preferably the amorphous ivermectin is in the form of particles with a particle size distribution ranging from about 0.1 μm to about 20 μm. If desired, the amorphous ivermectin may be provided in the form of particles with a particle size distribution ranging from about 0.5 μm to about 4 μm. In one preferred aspect, amorphous ivermectin may be provided in the form of particles wherein 90% of the particles have a particle size of less than 4 μm.

The invention provides amorphous ivermectin obtainable by, or obtained by, spray drying. This is a novel form of the compound. Suitably, the spray drying provides amorphous ivermectin in spherical particulate form.

In one aspect, the invention also provides amorphous ivermectin obtainable by, or obtained by, spray drying a solution of ivermectin in a solvent, wherein apart from ivermectin and solvent, the solution is free of any other ingredient.

In the method of the invention, the concentration of ivermectin in the solution is preferably from about 2% to about 30% by weight of the total weight of the solution; if desired the concentration of ivermectin in the solution may be from about 3% to about 7% by weight of the total weight of the solution.

In a preferred aspect, the amorphous ivermectin of the invention, or a pharmaceutical formulation thereof, or a medical device as described herein and containing amorphous ivermectin may be used in treating a medical condition caused by internal nematode infections including but not limited to onchocerciasis (river blindness), filariasis (elephantiasis), strongyloidiasis or demodicosis. In particular, they may be used for treating onchocerciasis (river blindness) in humans; or may be used in treating a medical condition caused by ectoparasitic infections including but not limited to infections caused by lice and mites.

In a preferred aspect, the invention provides amorphous ivermectin in isolated solid form obtained by spray drying a solution of ivermectin in a solvent, wherein the invermectin is in the form of particles having a particle size of less than 4 μm. Suitably the amorphous ivermectin is not in the form of a solid dispersion. It suitably comprises the drug alone, free of any other ingredients, other than any residual solvent(s) used during manufacture.

We have found that the water solubility for the amorphous form of ivermectin is higher than its corresponding crystalline form. According to our measurements, the known crystalline form shows no visible peak on the HPLC chromatogram (below the LOD of the method) while the amorphous form shows a small, but visible peak on the HPLC chromatogram. This confirms that the amorphous form is more water soluble than the known crystalline form.

Ivermectin can be dissolved in any suitable solvent (organic or aqueous) or mixtures thereof, such as ethanol or methanol and the solvent can be safely removed in a spray drying equipment. Any form of ivermectin may be used as the starting material. For example, crystalline ivermectin may be used. Other suitable solvents include for example methyl ethyl ketone, acetone, or 1-butanol. Preferably, a suitable solvent is one that would be kept below the defined ICH limits in the final product. Ethanol is used in the example shown below and has an ICH limit of 5000 ppm. In the method of the invention, the solvent may be any suitable solvent, but is preferably an organic solvent, or a mixture of organic solvents, such as those described above. In a preferred aspect, the solvent is such that a clear solution of ivermectin is obtained, that is, the ivermectin is completely dissolved.

When forming the amorphous material, any appropriate ivermectin concentration can be used. However, a solution concentration between 2% and 30% w/w is preferred, ideally 5% w/w where “% w/w” refers to the mass of the compound of formula [1] as a percentage of the mass of the total solution. The concentration to be employed will generally be limited by the solubility of ivermectin in the solvent of choice. The solution may, if desired, or required, be purified. This may be done before spray drying. Purification may be done using any suitable purification technique, as will be understood. For example, a filtration step, or an absolute filtration step, may be included. This may be, for example, be immediately before the final product isolation (by spray drying). Such a step may be included, for example, to remove foreign particles and control microbial and endotoxin contamination of the final API.

Spray drying may be performed using any suitable or commercially available equipment, and is our preferred method for forming the amorphous ivermectin of the invention.

A variety of atomization methods can be used, depending on the equipment being used. For example, a pneumatic spray nozzle orifice of 0.7 mm is suitable although alternative atomization methods such as rotary, pressure and ultrasonic nozzles may be employed.

The preferential atomization gas flow in terms of liters per hour can be adjusted to the equipment in use and any suitable atomization gas flow can be used. Typically, for a small-scale unit, 150 to 300 milliliters per hour is preferred. In an industrial scale a different flow may be used. In a preferred embodiment, the nozzle assembly can be cooled with a suitable fluid during spray drying to minimize product degradation.

Any suitable drying temperature can be used. In one aspect of this invention, the outlet temperature range may be from 20° C. to 100° C., preferably 30° C. to 50° C. and more preferably 40° C. to 45° C.

The inlet temperature may be adjusted to attain the desired outlet temperature.

Any suitable solution flow rate can be used. For a small-scale, the solution flow rate may preferably be from 1 to 20 ml/min, more preferably 2 to 15 ml/min for the 0.7 mm nozzle. For the industrial scale, the solution flow rate may be adjusted depending on the selected nozzle.

The drying gas flow rate for a small-scale spray dryer may be from about 20 kg/h to about 120 kg/h, preferably from about 40 kg/h to about 80 kg/h, most preferably about 40 kg/h. The drying gas flow rate for a larger spray dryer may be greater than about 120 kg/h preferably about 360 kg/h, about 650 kg/h or about 1250 kg/h.

In a particularly preferred aspect, the outlet temperature, atomization flow rate, solution concentration and solution flow rate, among other tested parameters, can be combined, as will be clear to one skilled in the art, to obtain compound [1] with a suitable quality.

The compound [1] obtained using the method of this invention is an amorphous solid. In one aspect of this invention, the amorphous solid is stable at room temperature (typically 25° C.) and at refrigerated temperatures (for example, at or below 3° C.).

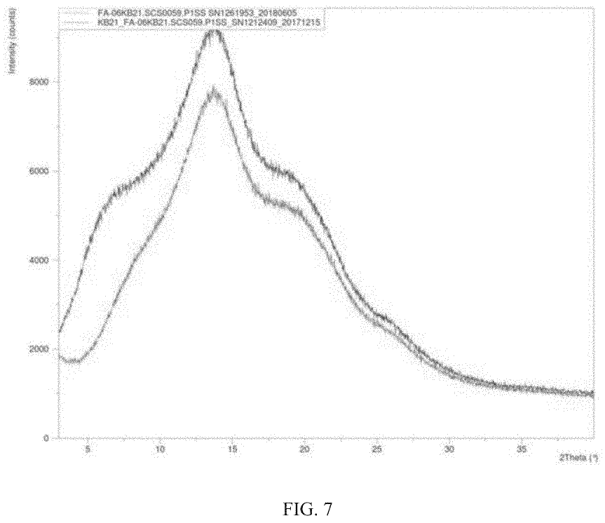

Unlike many amorphous materials, the amorphous ivermectin provided by the preset invention shows excellent stability. By stable we mean that the amorphous form is maintained after a certain period of time under certain environmental conditions. We have data confirming that the product maintains its amorphous form at room temperature for at least 3.5 months. Our stability data for the product shows that the amorphous form is stably maintained (without loss of amorphous character or transformation to another form) for at least 3 months in the freezer followed by at least 3.5 months at room temperature. This is illustrated, for example, in FIG. 7, which shows that the amorphous character is maintained before (blue, upper trace) and after (red, lower trace) storage for 3 months in the freezer followed by 3.5 months at room temperature (25° C.).

Particulate amorphous compound can be obtained directly from the spray dryer. Particles obtained may have a particle size ranging from 0.1 to 50 μm, typically with 90% of the particles below 10 μm. In one preferred aspect of this invention, 90% of the particles can be obtained below 4 μm.

The amorphous ivermectin of the present invention may be formulated into a range of pharmaceutical formulations in accordance with known techniques using suitable pharmaceutical excipients or carriers (depending upon the type of formulation) as will be understood by those skilled in the art. Generally speaking, the formulations known for crystalline ivermectin may be used, with appropriate modification, for amorphous ivermectin. Such formulations, or indeed amorphous ivermectin itself, may also be incorporated into a range of medical devices (for example, inhalers) as will also be understood by those skilled in the art.

### X-Ray Powder Diffraction Characterization

The aim of the X-ray powder diffraction analysis was to characterize the molecular arrangement of ivermectin obtained. The XRP diffractogram obtained is shown in FIG. 2.

The X-ray powder diffraction pattern of ivermectin obtained by spray drying according to the process herein disclosed is presented in FIG. 2.

X-ray diffraction (XRPD) analysis was made with PANalytical Empyrean equipped with copper tube and PIXel1D-Medpix3 detector (Malvern Panalytical, UK).

The XRPD diffraction pattern present halos which are characteristic of an amorphous material.

Differential scanning calorimetry (DSC) of the amorphous compound of formula [1] obtained by spray drying shows a glass transition temperature (Tg) of 136° C.

DSC measurements were performed using a Q2000 DSC (TA instruments, Waters, LLC, USA) at a heating rate of 10° C./min, between 25° C. and 350° C. Data collection and analysis were performed using TA Instruments Trios software.

### EXAMPLES

Example 1 is set forth to aid in understanding the invention but is not intended to, and should not be considered to, limit its scope in any way. The experiment reported was carried out using a BUCHI model B-290 advanced spray dryer.

### Example 1: Amorphous Ivermectin Isolation by Spray Drying

### Ivermectin Solution Preparation

Ivermectin in a mass proportion 5% (w/w), was dissolved in absolute of ethanol in a mass proportion of 5% until a clear solution was obtained.

### Isolation of the Particles

A lab scale spray dryer (Büchi, model B-290), equipped with a two fluid nozzle was used to atomize and dry the solution. Co-current nitrogen was used to promote the drying after atomization. The spray drying unit was operated in open cycle mode (i.e., without recirculation of the drying gas). Error! Reference source not found. schematically describes the spray drying set up used.

Before feeding the solution to the nozzle, the spray drying unit was stabilized with nitrogen and a solution of absolute ethanol to assure stable inlet (T_in) and outlet temperatures (T_out). After stabilization, the solution was fed to the nozzle by means of a peristaltic pump, and atomized at the tip of the nozzle. The droplets were then dried in the spray drying chamber by co-current nitrogen. The stream containing the dried particles was directed into a cyclone and collected at the bottom. The main operating parameters during the spray drying process are summarized in Table 1.

TABLE 1
Summary of the main operating conditions
Ivermectin solution parameters

| | | |
|---|---|---|
| Ivermectin | G | 25 |
| Absolute ethanol | mL | 602 |

Spray Drying parameters

| | | |
|---|---|---|
| T_in | ° C. | 60 |
| T_out | ° C. | 42 |
| F_drying (N₂) | kg/h | 40 |
| Rotameter level | Mm | 40 |

### Scanning Electron Microscopy Evaluation of the Spray Dried Material

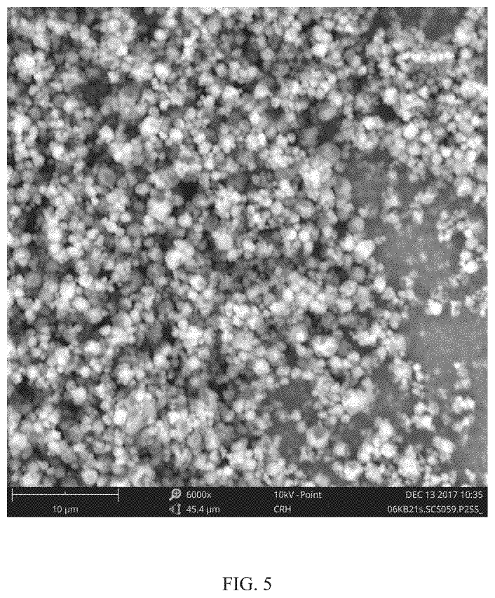

The particle size of the atomized material was characterized by means of scanning electron microscopy and laser diffraction (Sympatec). A representative image of the particles obtained is shown in FIG. 5. Generally, particles with a diameter of about 0.5-4 μm were seen.

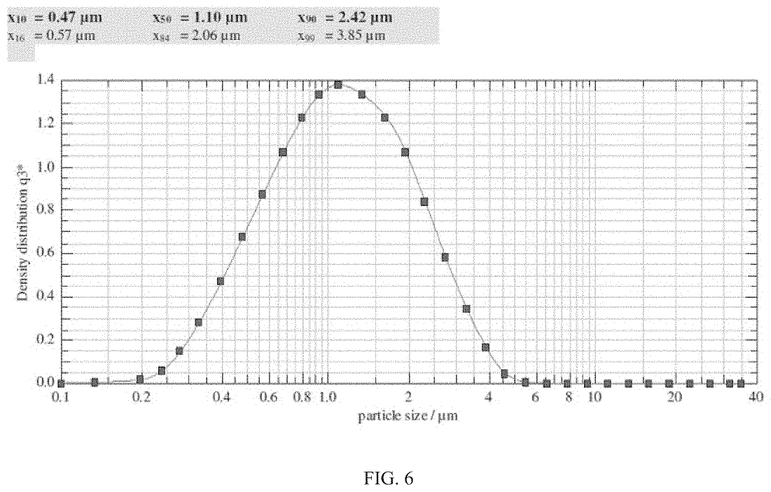

A representative histogram of the particle size distribution obtained is shown in FIG. 6. Generally, particles with a diameter of approximately 0.5-4 μm were obtained.

The usual particle size distribution (PSD) parameters (Dv10, Dv50, Dv90) are represented as ×10, ×50 and ×90.

1-9. (canceled)

10. Amorphous ivermectin in the form of particles with a particle size distribution ranging from 0.1 μm to 20 μm.

11. Amorphous ivermectin according to claim 10 in the form of particles with a particle size distribution ranging from 0.5 μm to 4 μm.

12. Amorphous ivermectin according to claim 10 in the form of particles wherein 90% of the particles have a particle size of less than 4 μm.

13. Amorphous ivermectin according to claim 10 obtainable by spray drying.

14. Amorphous ivermectin according to claim 10 obtainable by spray drying a solution of ivermectin in a solvent, wherein apart from ivermectin and solvent, the solution is free of any other ingredient.

15. A pharmaceutical formulation, for animal including human or veterinary use, comprising amorphous ivermectin according to claim 10, and a pharmaceutically-acceptable carrier therefor.

16. A medical device incorporating amorphous ivermectin according to claim 10, or incorporating a pharmaceutical formulation for animal including human or veterinary use, comprising amorphous ivermectin according to claim 10, and a pharmaceutically-acceptable carrier therefor.

17. A method of preparing amorphous ivermectin according to claim 10, which method comprises the steps of preparing a solution of ivermectin in at least one solvent; removing the solvent by feeding the solution to a spray dryer and collecting particles of ivermectin.

18. The method according to claim 17 wherein the solvent is an organic solvent or an aqueous solvent or mixtures thereof; a mixture of organic solvents, or water.

19. The method according to claim 17 wherein the concentration of ivermectin in the solution is from about 2% to about 30% by weight of the total weight of the solution.

20. The method according to claim 19 wherein the concentration of ivermectin in the solution is from about 3% to about 7% by weight of the total weight of the solution.

21. A method comprising utilizing amorphous ivermectin according to claim 10, or a pharmaceutical formulation for animal including human or veterinary use, comprising amorphous ivermectin according to claim 10, and a pharmaceutically-acceptable carrier therefor, as a medicament.

22. A method comprising utilizing amorphous ivermectin according to claim 10, or a pharmaceutical formulation for animal including human or veterinary use, comprising amorphous ivermectin according to claim 10, and a pharmaceutically-acceptable carrier therefor, in treating a medical condition caused by internal nematode infections including but not limited to onchocerciasis (river blindness), filariasis (elephantiasis), strongyloidiasis or demodicosis.

23. A method comprising utilizing amorphous ivermectin according to claim 10, or a pharmaceutical formulation for animal including human or veterinary use, comprising amorphous ivermectin according to claim 10, and a pharmaceutically-acceptable carrier therefor, in treating onchocerciasis (river blindness) in humans.

24. A method comprising utilizing amorphous ivermectin according to claim 10, or a pharmaceutical formulation for animal including human or veterinary use, comprising amorphous ivermectin according to claim 10, and a pharmaceutically-acceptable carrier therefor, in treating a medical condition caused by ectoparasitic infections including but not limited to infections caused by lice and mites.

25. Amorphous ivermectin in isolated solid form characterized by having an XRPD pattern as shown in, or substantially as shown in, FIG. 2 and obtained by spray drying a solution of ivermectin in a solvent, wherein the invermectin is in the form of particles having a particle size of less than 4 μm.
